(19) United States
(12) Patent Application Publication
(10) Pub. No.: US 2015/0080709 A1
(43) Pub. Date: Mar. 19, 2015

(54) Implantable Medical Devices, Methods of Use, and Apparatus for Extraction Thereof

(71) Applicant: Neha Chaturvedi, Shawnee, KS (US)

(72) Inventor: Neha Chaturvedi, Shawnee, KS (US)

(21) Appl. No.: 14/317,776

(22) Filed: Jun. 27, 2014

Related U.S. Application Data
(60) Provisional application No. 61/840,138, filed on Jun. 27, 2013.

(51) Int. Cl. A61B 5/06; A61B 17/3205; A61N 1/05

(52) U.S. Cl. CPC A61B 5/065; A61N 1/05; A61B 17/3205
USPC 600/424; 607/119; 607/120; 606/129

(57) ABSTRACT

One aspect of the present disclosure relates to an implantable medical device. The implantable medical device can include a main body portion having at least one photosensitive nanoparticle associated therewith. Delivery of energy to the main body portion promotes extraction of said implantable medical device from a subject.

### CROSS REFERENCE TO RELATED APPLICATIONS

This application claims priority to U.S. Provisional Application Ser. No. 61/840,138 filed Jun. 27, 2013, the entire contents of which is incorporated herein by reference.

### STATEMENT REGARDING FEDERAL FUNDING

Embodiments of the present invention were not conceived or developed with Federal funding or sponsorship.

### TECHNICAL FIELD

The present disclosure relates generally to devices and methods for removal of an implanted object from a subject's body, and more particularly to devices and methods for removal of endocardial leads from a patient's body using selected application of energy to the leads.

### BACKGROUND

Endocardial leads are often placed in contact with the endocardial tissue by passage through a venous access, such as the subclavian vein or one of its tributaries. Thus a transvenous endocardial lead refers to a pacemaker lead which contacts endocardial tissue through a vein. In the past, various types of transvenous endocardial leads have been introduced into different chambers of the heart including the right ventricle, right atrial appendage and atrium as well as the coronary sinus. These leads usually are composed of an insulator sleeve that contains a coiled conductor having an electrode tip attached at the distal end. The electrode tip is held in place within the trabeculations of endocardial tissue. The distal ends of many available leads include flexible tines, wedges, or finger-like projections which extend radially outward and usually are molded from and integral with the insulator sleeve of the lead. These tines allow better containment by the trabeculations of endocardial tissue and help prevent dislodgement of the lead tip.

Once an endocardial lead is implanted within a chamber, the body's reaction to its presence furthers its fixation within the heart. Specifically, shortly after placement, i.e., acute placement, a blood clot forms about the flanges or tines due to enzymes released in response to the irritation of the endocardial tissue caused by electrode tip. Over time, i.e., during chronic implantation, fibrous scar tissue eventually forms over the distal end, usually in three to six months. In addition, fibrous scar tissue often forms, in part, over the insulator sleeve within the venous system and the heart chamber. Such tissue fixes the electrode tip within the heart during the life of the lead.

Although the state of the art in implantable pulse generator or pacemaker technology and endocardial technology has advanced considerably, endocardial leads nevertheless occasionally fail, due to a variety of reasons, including insulation breaks, breakage of the inner helical coil conductor thereof and an increase in electrode resistance. Also, in some instances, it may be desirable to electronically stimulate different portions of the heart than that being stimulated with leads already in place. Due to these and other factors, therefore, a considerable number of patients may come to eventually have more than one, and sometimes as many as four or five, unused leads in their venous system and heart.

Unused transvenous leads increase the risk complications will develop. Possible complications associated with leaving unused leads in the heart and venous system include an increased likelihood an old lead may be the site of infection. Development of an infection may, in turn, lead to septicemia, a possibly fatal complication. Unused leads may also cause endocarditis. Furthermore, unused leads may entangle over time, thereby increasing the likelihood of blood clot formation. Such clots may embolize to the lung and produce severe complications or even fatality. The presence of unused leads in the venous pathway and inside the heart can also cause considerable difficulty in the positioning and attachment of new endocardial leads in the heart. Moreover, multiple leads within a vein or artery may impede blood flow causing fatigue, weakness or dizziness within the patient.

### SUMMARY OF THE INVENTION

Embodiments of the present invention are directed to an implantable device, an extraction device and a method of using such implantable device and extraction device to extract the implantable device with a minimum of trauma to the animal or human in which the implantable device is placed. One embodiment directed to the implantable device for implantation into tissues of an animal or human comprises at least one body having an exterior surface for placement in intimate contact with a non-fluid tissue in which the device is implanted. The device further comprises at least one of the photoreactive agent, selected from the group consisting of a photo sensitizer or photosensitive nanoparticle, associated with the exterior surface. The delivery of light energy to the photoreactive agent generates at least one of the following forms of energy consisting of reactive compounds or thermal energy and promotes release of the exterior surface from the tissues in which it is implanted.

As used herein, the term “non-fluid tissue” refers to soft tissues, such as muscle, skin, fat and the like, and hard and semi hard tissues such as cartilage and bone, which tissues tend to form adhesions to foreign materials, when such foreign objects are placed into immediate direct contact with the tissue. The term is intended to exclude blood tissues. The term “intimate” is used in the context of such immediate and direct contact.

Embodiments of the present invention feature a photosensitive nanoparticle. The term “photosensitive nanoparticle” refers to particles and the like as taught and suggested by Canadian reference CA239929, entitled Optically Active Nanoparticles for Use in Therapeutics and Diagnostic Methods, the entire contents of which is incorporated herein by reference. The photosensitive nanoparticle generates thermal energy at the exterior surface and the tissue surrounding the exterior surface;

The implantable device of the present invention has a body associated with the group consisting of pacemakers, defibrillators, nephrotomy tubes, indwelling vascular catheters, indwelling neural brain stimulators, indwelling structures, surgical mesh, cochlear implants, dental implants, bladder stimulators, intracardiac monitoring devices, and indwelling stents. The body itself or some part of the implantable device presents a surface that is intimately in contact with the tissue in which it is implanted. For example, without limitation, the detailed description will describe a body comprising a pacemaker and an exterior surface comprising the cardiac pacing lead of the pacemaker.

One embodiment features an implantable device wherein the at least one photosensitive nanoparticle is constructed and arranged in association with the exterior surface to cooperate with a source of light energy, to receive the light energy about the exterior surface in intimate contact with the tissue. That is, the at least one nanoparticle is positioned about the exterior surface so that the nanoparticle will receive the photons from a source.

One embodiment features an exterior surface having a therapeutic agent. The therapeutic agent is associated with the nanoparticle or independent of the nanoparticle or both. For example, without limitation, one therapeutic agent is an anti-infective agent. Nanoparticles and therapeutic agents are placed on coatings on the implantable device. Coating are available from multiple sources including SurModics, Inc. (Eden Prairie, Minn., USA), Helix Medical (Carpinteria, Calif. USA). See also: Franck Furno et al., Silver nanoparticles and polymeric medical devices: A new approach to prevention of infection, J. Antimicrob, Chemother, (December 2004) 54 (6): 1019-1024

One embodiment features a photosensitive nanoparticle responsive to a selected wavelength of light. The nanoparticle is substantially non-responsive or inert to all other wavelengths so the activation of the nanoparticle can be controlled. For example, without limitation, the selected wavelength of light is produced by a laser.

A further embodiment of the present invention is directed to an extraction device for use with an implantable device described above. The extraction device has an extraction housing having a first end and a second end. The first end has means for manipulation by an operator and the second end is constructed and arranged for entering an animal or human subject and being positioned proximal to the exterior surface. The second end has a photon emitting means for producing light energy. The light energy is received by the photosensitive nanoparticle and the photosensitive nanoparticle generates thermal energy which promotes release of the exterior surface from the tissues in which it is implanted.

One embodiment features an extraction device wherein said extraction housing is a catheter. One embodiment of the catheter has a guide lumen. And, the photon emitting means is an optical fiber. The optical fiber is in optical communication with a laser or is constructed and arranged to be connected in optical communication with a laser. One embodiment features a plurality of optical fibers arranged in a circle about said second end to deliver light around said exterior surface.

One embodiment of the extraction device features a second end comprises cutting means. One cutting means is a substantially circular cutting edge constructed and arranged larger than the exterior surface to allow the cutting edge to cut around the exterior surface.

One embodiment of the extraction device features an extraction sheath for enveloping the exterior surface. The sheath is extendable from the catheter and is retrievable, that is capable of being withdrawn into the catheter lumen holding or engaging the exterior surface.

One embodiment of the extraction device further comprises a port in fluid communication with one or more liquids to allow administration of such liquids to the location of the exterior surface. For example without limitation, liquids having light quenching compounds may be used to contain or absorb spurious light energy. Or, the liquids may comprise anti-infective agents or anti-inflammatory agents.

A further embodiment of the present invention is directed to a method of extracting an implanted device which is implanted into tissues of an animal or human. The implanted device has at least one body having an exterior surface placed in intimate contact with a non-fluid tissue. The implanted device further comprising at least one photoreactive agent associated with the exterior surface. And, delivery of light energy to the photoreactive agent promotes release of said exterior surface from the tissues in which it is implanted. The method comprises the step of identifying the exterior surface of the implanted device. And, the method comprises the step of applying light energy about at least one of the exterior surface and the non-fluid tissue where the implanted device is located, promoting the release of the exterior surface from the tissue

As used herein, the term “identifying” means locating the position of the exterior surface to which light energy will be applied.

One embodiment features a photosensitive nanoparticle in thermal communication with at least one of the group consisting of said exterior surface and the tissue surrounding said exterior surface.

Embodiments of the present method have utility for extracting an implanted device where the body is selected from the group consisting of pacemakers, defibrillators, nephrotomy tubes, indwelling vascular catheters, indwelling neural brain stimulators, indwelling structures, surgical mesh, cochlear implants, dental implants, bladder stimulators, intracardiac monitoring devices, and indwelling stents. The detailed description describes a pacemaker and an exterior surface is on a cardiac pacing lead.

One embodiment of the present method features at least one photosensitive nanoparticle constructed and arranged in association with the exterior surface to cooperate with a source of light energy, to receive said light energy about the exterior surface in intimate contact with the tissue.

Another embodiment features an exterior surface having a therapeutic agent. The therapeutic agent may be coupled to the nanoparticle to be released by thermal energy or may be carried separately and apart of the nanoparticle as a separate coating. One embodiment of the method features a therapeutic agent comprising one or more of the groups of compounds consisting of anti-infective agents, anti-inflammatory agents and quenching agents.

One embodiment of the present method features at least one photosensitive nanoparticle responsive to a selected wavelength of light. The method further features a selected wavelength of light produced by a laser.

One embodiments of the present method features an extraction device. The extraction device is used to remove the implanted device. The extraction device has an extraction housing having a first end and a second end. The first end has means for manipulation by an operator and said second end is constructed and arranged for entering an animal or human subject and being positioned proximal to the exterior surface body. The second end has a light emitting means for producing light energy. The light energy is received by the photoreactive agent, which promotes the release of the exterior surface from the tissues in which it is implanted. The method further comprises the steps of positioning the second end proximal to the exterior surface and using the light emitting means to promote the release of said exterior surface.

One embodiment of the present invention features a photosensitive nanoparticle which generates thermal energy upon receiving light energy.

One embodiment of the present method features an extraction housing comprising a catheter. The catheter has a guide lumen which the operator manipulates from the first end. The catheter has light emitting means in the form of an optical fiber. One embodiment features an optical fiber in optical communication with a laser.

One embodiment of the present method features light emitting means as a plurality of optical fibers arranged in a circle about the second end to deliver light around said exterior surface.

A further embodiment features a second end further comprises cutting means and the additional step of cutting the exterior surface free of the tissue. One method features cutting means having a substantially circular cutting edge constructed and arranged larger than said exterior surface to allow said cutting edge to cut around the exterior surface.

A further embodiment of the present method features a catheter having an extraction sheath for enveloping the exterior surface. The method comprises the steps and of surrounding the exterior surface with the sheath to grip the exterior surface and withdraw the exterior surface from surrounding tissue.

One embodiment of the present invention features a catheter having a port in fluid communication with one or more liquids to allow administration of such liquids to the location of the exterior surface. The method further comprises the step of irrigating said exterior surface with one or more liquids from the port.

These and other features and advantages will be apparent to those skilled in the art upon viewing the drawings which are described briefly in the text below and upon reading the Detailed Description that follows.

### BRIEF DESCRIPTION OF THE DRAWINGS

FIG. 1 is a schematic view of a pacemaker/defibrillator device having a lead having an exterior surface implanted in the heart tissue of an individual;

FIG. 2A depicts the lead having an exterior surface;

FIG. 2B depicts a cross section of the lead;

FIG. 3 depicts the lead implanted in the right ventricular myocardium;

FIG. 4 depicts the lead implanted in the right atrium;\

FIG. 5A depicts a cut section of the lead of FIG. 1;

FIG. 5B depicts a cross section of the lead of FIG. 1;

FIG. 6 depicts a cross section of a photosensitive nanoparticle of nano shell;

FIG. 7A depicts an extraction device in accordance with the present invention;

FIG. 7B depicts an extraction device in accordance with the present invention;

FIG. 7C depicts an extraction device in accordance with the present invention;

FIG. 8A depicts an extraction device in accordance with the present invention;

FIG. 8B depicts an extraction device in accordance with the present invention;

FIG. 9A depicts an extraction device positioned about a lead in accordance with the present invention;

FIG. 9B depicts an extraction device positioned about a lead in accordance with the present invention;

FIG. 10A depicts an extraction device positioned about a lead in accordance with the present invention;

FIG. 10B depicts an extraction device positioned about a lead in accordance with the present invention.

### DETAILED DESCRIPTION OF THE INVENTION

The present disclosure relates generally to devices and methods for removal of an implanted object from a subject's body, and more particularly to devices and methods for removal of endocardial leads from a patient's body using selected application of energy to the leads.

One aspect of the present disclosure relates an implantable medical device. The implantable medical device can include a main body portion having at least one photosensitive nanoparticle associated therewith. Delivery of energy to the main body portion promotes extraction of said implantable medical device from a subject.

Another aspect of the present disclosure relates to a method for extracting an implanted medical device from a subject. One step of the method includes identifying a medical device implanted in the subject. The medical device can comprise a main body portion including at least one photosensitive nanoparticle and/or photosensitizer associated therewith. Next, an amount of energy is applied to at least a portion of the main body portion for a time sufficient to cause the implanted medical device to substantially detach from the surrounding bodily tissue. The medical device is then removed from the subject without damage to the bodily tissue surrounding the medical device.

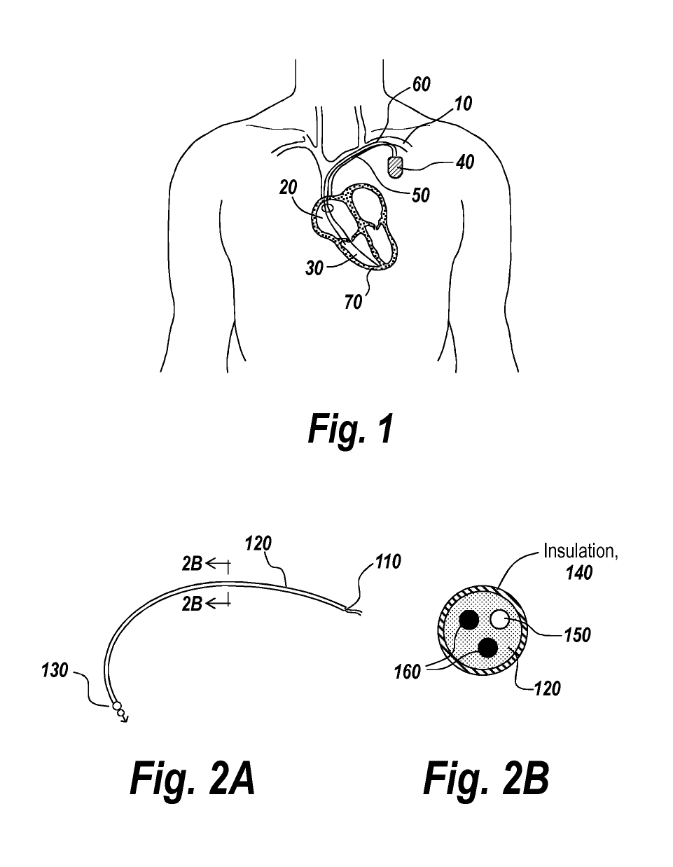

Turning now to FIG. 1 shows schematic representation of defibrillator/pacemaker implanted in situ according to one aspect of the present disclosure. The pacemaker generator (40) is attached to the leads going into the atria (60) and lead going into right ventricle (50). The leads are inserted via the subclavian vein (10) and the tips are inserted into right atria (20), right ventricle (30) and its apex (70);

FIG. 2 shows the lead of FIG. 1 in its longitudinal form and its cross-section. The electrodes (110) are encased inside the lead body (120), which terminates at the tip (130). The cross-section shows electrodes (160) and a guidewire lumen (150) encased in a lead material (170) and covered with an insulation covering (140);

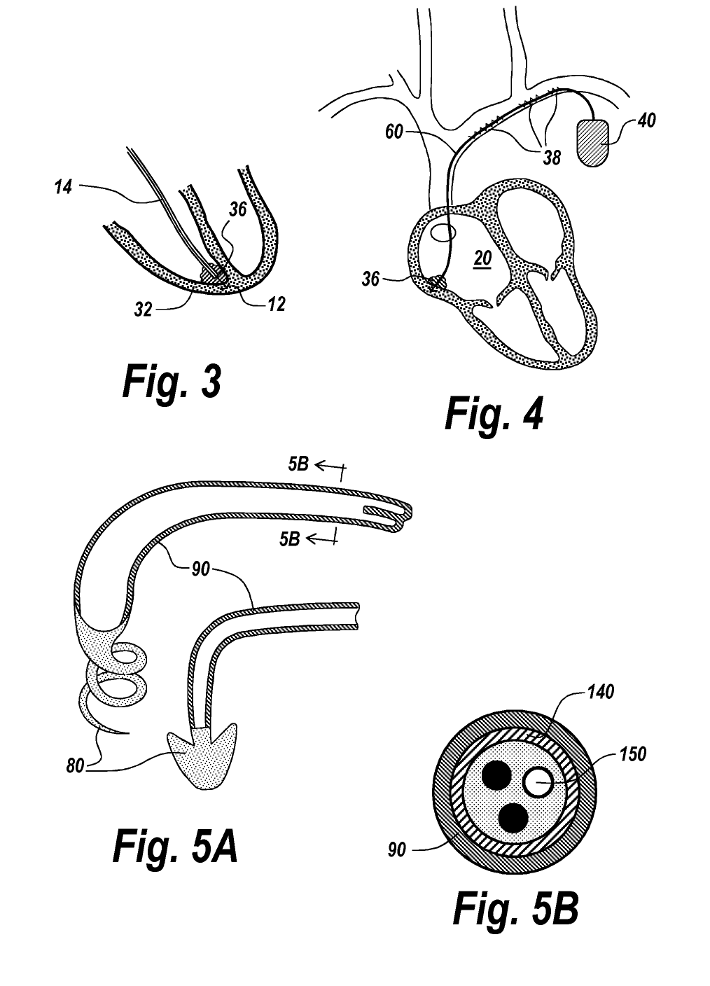

FIGS. 3-4 show a tip of the lead in FIG. 1 implanted in the right ventricular myocardium and right atrium. FIGS. 3-4 also show the adhesive reaction and attachment of lead to the myocardium near its tip (36) and adhesions (38) in its course in the subclavian vein (10) and superior vena cava (60);

FIG. 5 shows a cut section and cross-section of the lead in FIG. 1 with a coating material. A coating (90) containing a photosensitizer or photosensitive nanoparticle covers all surfaces in the lead described in FIG. 2;

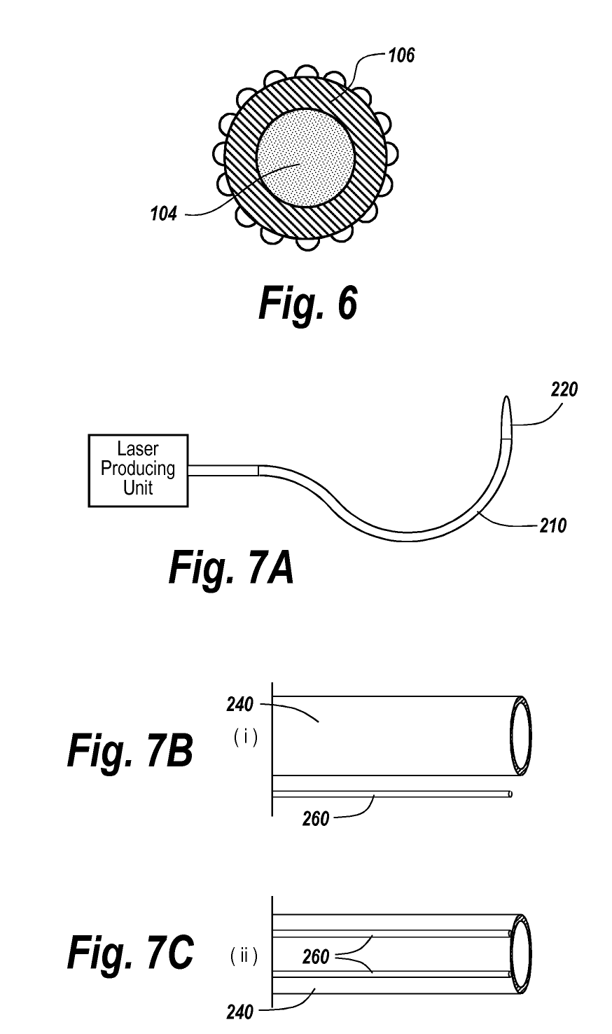

FIG. 6 shows a nanoshell with a silica core (104) and a gold shell (160);

FIG. 7 shows the basic diagram of laser extraction device with a laser producing unit, a catheter with at least one optical fiber (210) and a terminal probe (220). Two optional configurations of the terminal probe are shown. Configuration (i) shows a cylindrical extraction tool (240) with sharp edges and a thin diameter catheter/optical fiber (260) inserted separately into the lead to be extracted. Configuration (ii) shows the extraction tool (240) with the optical fibers (260) arranged on the circumference of the extraction tool;

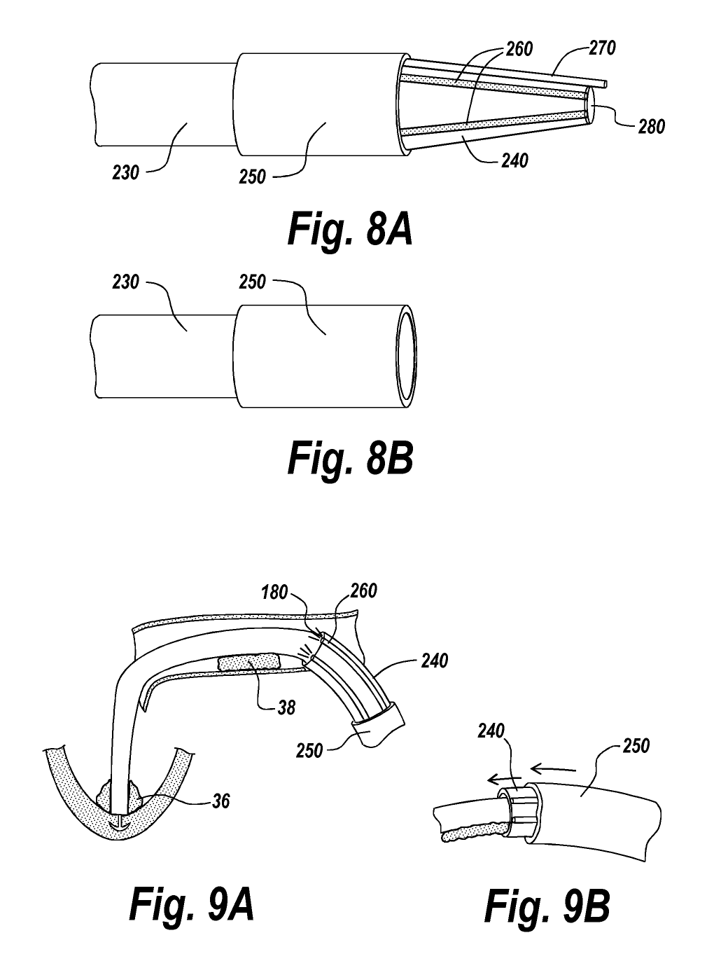

FIGS. 8A-B show open (sliding out) and closed positions of a terminal extraction probe (respectively) according to another aspect of the present disclosure. The shaft of the sheath (230) is attached to the assistant tool (250), and the extraction tool (240) can slide through the assistant tool in the open position (a). The optic fibers (260) are attached to the extraction tool at its circumference and a lens (280) can be attached to its tip to focus the laser beam. Optionally, there is a port (270) attached to the extraction tool, which can deliver coating materials or flush to the extraction site in situ;

FIG. 9 shows an extraction method according to another aspect of the present disclosure. The laser beam is delivered from the optical fiber (260) or the lens mechanism (280) which activates the coating. The extraction tool and the assistant tools are then advanced over this lead thus creating a space between the lead and adhesions/surrounding structures. This unit is advanced until the probe reaches the tip of the device; and

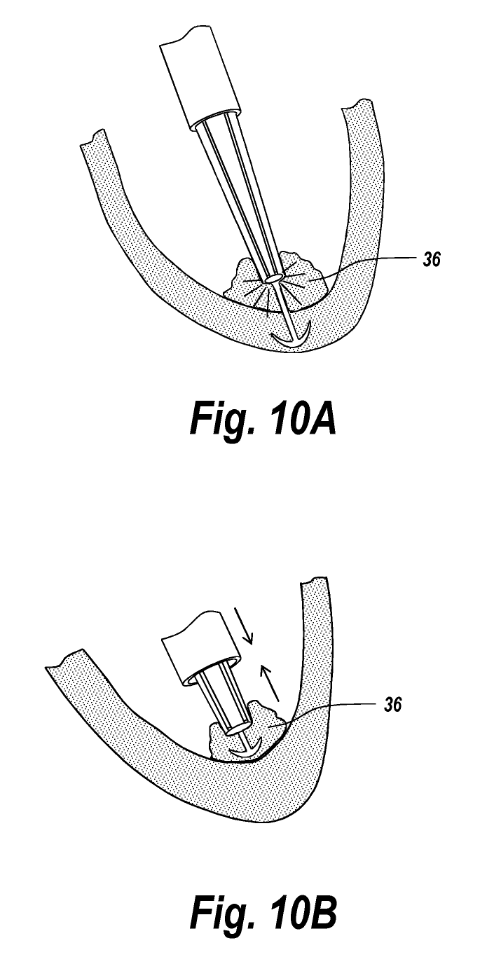

FIG. 10 shows the terminal probe in FIGS. 8A-B while performing extraction near the tip of lead at the myocardial insertion point. FIG. 10 also shows laser beam activating the coating of the device and detaching from the adhesions (36). Closing the device, such as by retraction of extraction tool and advancing assistant tool simultaneously, may lead to extraction of tip from myocardium.

Unless defined otherwise, all technical and scientific terms used herein have the same meaning as is commonly understood by one of skill in the art to which the present disclosure pertains.

### Overview

One aspect of the present disclosure relates to medical devices, and more particularly to implantable medical devices that remain inside the body for prolonged periods of time. Such medical devices, as understood herein, can include lead of pacemaker or defibrillator device, nephrostomy tubes, indwelling vascular catheters, indwelling neural/brain stimulators, spinal cord stimulators, indwelling structures (e.g., inferior vena cava filters), surgical mesh, cochlear implants, dental implants, bladder stimulators, intracardiac monitoring devices (e.g., implantable MEMS devices), indwelling stents (e.g., J-stents, ureteral stents, bladder stents, coronary stents, and stents in peripheral arteries. Medical devices can also include other devices that stay inside body for diagnostic indications, therapeutic indications, or both. In such instances, medical devices can be left inside the body and be in contact with different biological surfaces (e.g., bodily fluids, tissues and artificial grafts).

In one example, an implantable device according to the present disclosure can include a device is a pacemaker/defibrillator device lead that is implanted into the right ventricle or the coronary sinus through a venous access system (e.g., the superior vena cava). The lead can be left in place either via a screw-in mechanism or other such device so that the lead is not displaced with motion and other activities. Over time, there is a fibrous reaction to the lead from adjoining structures, which cause adhesions and fibrous strands to get attached to the tissue and the external surface of the lead. There may also be growth of an endothelial layer of the vascular tissue or the heart over the lead, thereby encasing it and attaching the lead to the adjacent structure(s).

Most of the time, these adhesions do not lead to problems; however, such devices may occasionally develop a complication necessitating its removal (e.g., lead dysfunction, fractures, infections or other issues). Lead removal is a complicated procedure, which is done either by open surgery or percutaneously by performing a procedure in cardiac laboratory. Several devices have been designed to perform this procedure, such as blunt sheaths that go around or over the lead, laser removal sheaths, and adjustable rotating sheaths. Despite these advances, the procedure of lead extraction is still a high-risk procedure that involves such complications as perforation of vital structures (e.g., venous structures, such as the superior vena cava, right atrium, or right ventricular wall of the heart). Some of these complications can be fatal. Advantageously, the present disclosure provides implantable medical devices and related methods which make removal of implanted devices easier and safer.

### Implantable Devices

One aspect of the present disclosure includes an implantable medical device. The implantable medical device can include a main body portion having at least one photosensitive nanoparticle associated therewith. Delivery of energy to the main body portion promotes extraction of said implantable medical device from a subject.

In some instances, all or only a portion of the main body portion can be contacted, impregnated, coated, or otherwise physically associated with a composition that includes at least one photosensitive nanoparticle. As explained in more detail below, providing implantable medical devices with photosensitive nanoparticles can achieve prevention or treatment of infection, an increased bio-compatibility of the medical device, localized periodic therapeutic agent delivery, and improving medical device extraction.

An implantable medical device can be treated with one or a combination of the same or different photosensitive nanoparticles such that it is easier to extract the medical device at a later date by photoactivation of the nanoparticle(s). One such method can involve performing photodynamic therapy in which chemicals known as photosensitizers are impregnated in the outer covering of a medical device to be implanted. At time of extraction, the medical device can be exposed to energy (e.g., a laser) that activates the photo sensitizer to form reactive oxygen species, which cause localized damage to adjacent tissue and detachment from dense adhesions. Alternatively, using plasmonic photothermal ablation, the medical device can be treated with nanoparticles, which are then exposed to extrinsic energy (e.g., a laser) that converts light to heat energy locally and causes cellular damage to adjacent structures. Coating are available from multiple sources including SurModics, Inc (Eden Prairie, Minn., USA), Helix Medical (Carpinteria, Calif. USA). See also: Franck Furno et al., Silver nanoparticles and polymeric medical devices: A new approach to prevention of infection, J. Antimicrob, Chemother, (December 2004) 54 (6): 1019-1024. One can coat the terminal screw in the manner of the lead. The terminal portion may be formed by metallic screws or similar anchoring mechanism which is different in composition from the body of the lead. This portion will be coated differently by employing existing coating technologies such as direct coating of nano particle on the surface or embedded nano particle in polymers.

Other potential coating methods may utilize one of the following or the combination of; stabilization of thin nano particle coating by using ultrathin added coating of other materials such as silica (example; as suggested in the article by Yulia Chaikin et al., Stabilization of Metal Nanoparticle Films on Glass Surfaces Using Ultrathin Silica Coating, Anal Chem., 2013, 85 (21) pp 10022-10027) and as ultrasonic spray painting of nanoparticles available through Analytical Technologies Pte Ltd Singapore.

To prevent/treat infections associated with an implanted medical device, an implantable medical device can be treated with nanoparticles prior to implantation (e.g., at time of manufacturing) or can be added as a coating when an infection is present or suspected. The nanoparticles may be in the shape of nano-rods, nano-spheres, nano shells, or other such nano-sized particle that has a shell and a core. For the use of nanoparticle-coated leads to prevent and/or treat infection, for instance, a nanoparticle-treated lead can be inserted in the body. At time of activation, electromagnetic energy in the form of light or other radiation source (e.g., laser beams) are irradiated upon the lead to activate the nanoparticles, thereby leading to heat and cellular damage to adjacent infectious organisms. This energy could be delivered from a source through the lead. Optionally, the laser energy or radiation in the near infrared region of the spectrum could be delivered from outside the body for activation of nanoparticles.

Treating medical devices with photosensitive nanoparticles can also increase biocompatibility of the medical devices. Various indwelling or implantable medical devices are made to serve such a function inside the bodily tissues that they remain in contact with various tissues, such as fluids (e.g., blood), secretions, muscular, osseous, neural, or other such bodily tissues. Medical devices according to the present disclosure can be manufactured with an intention to reduce secretion of the device coating inside these tissues, and also not form adhesions or interfere with other normal function in situ. Upon residing in tissue, the photosensitive nanoparticles can increase biocompatibility by just forming a coating layer or more than one layer. In some instances, the nanoparticle coating can be activated at periodic intervals (e.g., days, weeks or months) by application of laser at a wavelength that causes activation of such a nanoparticle and light energy is converted to heat energy causing dissociation of attached debris, thereby increasing compatibility with bodily tissues. This laser application may be internal via an optic fiber or external by near infrared wavelength laser emission.

In other instances, implantable medical devices according to the present disclosure can be treated with a pharmaceutical agent. For example, nanoparticles can encase drugs such that when photoactivation occurs, local delivery of the drugs can be achieved. In such instances, drugs delivered may include antibiotics that can be employed in implantable intravascular catheters that communicate with external environments, such as central lines, nephrostomy tubes, urinary catheters, etc. It may also include drugs that inhibit the development of fibrosis locally. Such drugs may optionally include molecules known to reduce endothelialization, such as Sirolimus, Zoratolimus, Paclitaxel, and the like. To activate the nanoparticles and thus any drugs associated therewith, activation may be done at a time after implantations (e.g., hours, days or months) for localized drug delivery. This activation may be performed by laser activation from an outside source, which may optionally use ultrashort lasers in the near infrared region.

In some instances, implantable medical devices can include one or more photosensitizers (or polymer conjugates of these photosensitizers) that are enzyme activatable. For example, a photo sensitizer (or polymer conjugates of this photo sensitizer) can be used such that it is inactive (not photo-activable) in one form and, on application of certain enzymes, it may become active (or photo-activatable). Photosensitizers can be activated in a variety of ways depending upon the photosensitizer itself, including photodynamic therapy, plasmonic photothermal therapy using nanoparticles, such as nanoshells and nanorods, and photothermal therapy using other photothermal agents, such as indocyanine green.

Photodynamic therapy (“PDT”) employs compounds known as photosensitizers, which can be activated by electromagnetic waves to selectively target and destroy cells. PDT involves delivering electromagnetic wave (such as light) of the appropriate wavelength to excite the photosensitizer molecule. This excited state can then undergo further reaction by one or both of two pathways, known as Type I and Type II photoprocesses. The Type I pathway involves production of radical ions that can react with oxygen to form toxic species, such as superoxide, hydroxyl and lipid derived radicals. The Type II pathway involves production of excited state oxygen, which can then oxidize many biological molecules, such as proteins, nucleic acids and lipids, and lead to cytotoxicity.

Photosensitizers that may be used as part of the present disclosure can include photofrin, synthetic diporphyrins and dichlorins, phthalocyanines, derivatives of phthalocyanine, verdins, purpurins, hydroporphyrins, etiopurpurin, octaethylpurpurin, chlorins, chlorin e₆, derivatives of chlorin e₆, meta-tetrahydroxphenylchlorin, bacteriochlorins, some tetra(hydroxyphenyl)porphyrin, benzoporphyrin derivatives, benzoporphyrin monoacid derivatives, derivatives of benzoporphyrin, 3,1-meso tetrakis (o-propionamido phenyl)porphyrin, naturally occurring porphyrins, hematoporphyrin, hematoporphyrin derivatives, sulfonated aluminum PC, sulfonated AlPc, disulfonated, tetrasulfonated derivative, sulfonated aluminum naphthalocyanines, naphthalocyanines, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, pyropheophorbides and ether or ester derivatives, protoporphyrin, ALA-induced protoporphyrin IX, endogenous metabolic precursors, 5-aminolevulinic acid benzonaphthoporphyrazines, cationic imminium salts, tetracyclines, lutetium texaphyrin, tinetio-purpurin, porphycenes, benzophenothiazinium, pentaphyrins, texaphyrins and hexaphyrins, 5-amino levulinic acid, hypericin, pseudohypericin, hypocrellin, terthiophenes, azaporphyrins, azachlorins, rose bengal, phloxine B, erythrosine, iodinated or brominated derivatives of fluorescein, merocyanines, nile blue derivatives, pheophytin and chlorophyll derivatives, bacteriochlorin and bacteriochlorophyll derivatives, porphocyanines, benzochlorins and oxobenzochlorins, sapphyrins, oxasapphyrins, cercosporins, and related fungal derivatives.

Plasmonic Photothermal photoablation (“PPTP”) employs nanoparticles, such as nanospheres, nanoshells, nanodiscs or nanorods, or nano-sized particles in other shapes that can be activated by electromagnetic wave to convert photon into heat energy, thus causing cytotoxicity. When nanoparticles, such as gold nanospheres are exposed to electromagnetic waves, such as a laser, the electric field induces a collective oscillation of the surface electrons (surface plasmons) in strong resonance with its frequency, a process known as the surface plasmon resonance (SPR). At a particular wavelength of the electromagnetic spectrum, the SPR and photoabsorption is maximal for a given nanoparticle. When the nanoparticles are exposed to that wavelength, such as with laser, it absorbs photons and converts to heat energy. The local environment around the nanoparticle is thus overheated due to the conversion of light energy to heat, a phenomenon, which can be optimally designed by using light radiation (such as a laser) with a wavelength overlapping with the SPR wavelength of the nanoparticle. Thus, PPTP nanoparticles can be localized to the area of interest and laser energy delivered to cause a local increase in temperatures (e.g., up to 40-70 degree Celsius or more).

In one example, the nanoparticle utilized can have an SPR wavelength that overlaps with laser wavelength in near infrared region (e.g., 700-1000 nm).

Photothermal therapy employs similar principle of converting light energy into heat energy by photothermal agents including, but not limited to, indocyanine green and other such agents.

### Extraction Devices

Another aspect of the present disclosure can include an extraction device comprising a catheter having a guide lumen and at least one optical fiber associated with the catheter and being configured to emit laser light energy. A distal end portion of the catheter can include an annular cutting edge.

As discussed below, extraction devices of the present disclosure can be used to deliver energy (e.g., a laser) to an implanted medical device and facilitate detachment from surrounding structures. Although extraction devices are described below in terms of extracting a lead, it will be appreciated that such devices can be configured to accommodate and be used to extract other types of implantable devices, such as those discussed above.

FIG. 7 shows the basic functional diagram of an extraction device according to one aspect of the present disclosure. A laser source is connected to an apparatus/catheter that contains at least one optical fiber capable of transmitting laser to the terminal probe. The laser source may produce laser beams from a gas, chemical, dye, metal-vapor, solid-state, semiconductor or other types of laser source, which produces either fixed, or variable wavelength of laser. In some instances, a laser can include a solid-state laser source or gas laser source producing laser in the near infra-red (NIR) region of electromagnetic spectrum. Other lasers can include lasers with small pulse duration in the range of femto second called ultrashort lasers. Ultrashort lasers produced in the NIR spectrum may be transmitted in the human/animal tissue for several centimeters without causing much heating due to its short pulse duration.

The terminal probe may be designed in one, or a combination of configurations detailed in FIGS. 7-8. The basic components of the terminal probe includes at least one or more optical fiber capable of transmitting laser beams perpendicular to, parallel to, or at an angle to the orientation of the device to be extracted. The probe has a sharp edged circular “extraction tool” which is cylindrical in shape and which encases the lead to be extracted. In one example, the optical fibers are connected to the extraction tool in a circular fashion so that they can deliver laser energy around the lead. There is an optional “assistant tool”, which is a cylindrical tool and could optionally be placed around or inside the “extraction tool”. The function of this tool is to provide blunt force and move the extraction sheath over the lead to be extracted. This whole unit is mounted on top of an extraction sheath. In an optional design, the “extraction tool” has a small port that is connected to outside and can deliver small amounts of liquid material such as diluted “coating material” detailed above or enzyme, which can activate an inactive form of photo sensitizer, saline or water.

In one configuration (FIG. 7(i)), the extraction device has a tool which lays outside the lead and a thin catheter which houses at least one optical fiber which can be inserted in the central lumen (guidewire lumen) of the lead to be extracted. At time of extraction, the laser beam can be delivered from the central catheter/optical fiber perpendicular to or at an angle to the orientation of the lead, such that it activates the surface coating of the lead and causes detachment. The tool can be advanced in small steps from the distal most tip of the lead slowly towards the terminal screw in portion in the heart.

In another configuration (FIG. 7(ii), the optical fibers are arranged around at the edge of the “assistant tool” which delivers laser beams perpendicular to, parallel to or at an angle to the orientation of the lead to be extracted. The “extraction tool” can then be advanced over the lead slowly from the distal tip of the lead towards the terminal screw in portion towards the heart.

In yet another configuration (FIG. 8), the optical fibers are arranged around the edge of the “assistant tool” with an additional optical or other type of lens attached to the tip of at least one of the optical fiber in such a way as to focus the laser beam on to the adhesions outside the lead. This apparatus may optionally work by providing additional heat based detachment of some elements of adhesions outside the lead. Optionally, this apparatus with the “assistant tool” may also be made to rotate around the lead thus delivering laser beam in a circular fashion around the lead.

### Methods

Another aspect of the present disclosure can include a method for extracting an implanted medical device from a subject. One step of the method includes identifying a medical device implanted in the subject. The medical device can comprise a main body portion including at least one photosensitive nanoparticle and/or photosensitizer associated therewith. Examples of photosensitizers are discussed herein. Next, an amount of energy is applied to at least a portion of the main body portion for a time sufficient to cause the implanted medical device to substantially detach from the surrounding bodily tissue. The medical device is then removed from the subject without damage to the bodily tissue surrounding the medical device.

In some instances, if extraction of an implanted medical device is desired, energy can be delivered at such a wavelength so that it activates at least one of the photo sensitizer or photosensitive nanoparticle. This leads to localized heat/reactive oxygen species formation leading to detachment from fibrous scar tissue and endothelium. One such method would include passing a thin wire or optical fiber in the central lumen of the medical device (e.g., lead), which delivers the energy. In another method, a sheath is passed covering the lead and delivers energy. After removal of the skin and tissue layers covering the medical device, the device (e.g., lead) is freed from the generator chamber. The extraction device with the terminal probe in one of the above discussed configurations will be employed to extract the device as shown in FIGS. 9A, 9B, 10A and 10B.

If configuration (i) is employed, the central optical fiber unit is advanced in the lumen (guidewire lumen) of the lead, while the extraction tool is advanced over the lead. When resistance is encountered while advancing the extraction tool, the central optical fiber is switched to emit a laser beam, which activates the coating on the lead. As discussed earlier, the coating is likely to be applied prior to insertion of the lead. However, in older leads, which do not have the photosensitizer, an additional injection port in the extraction or assistant tool detailed above may optionally inject this photosensitizer as detailed above.

Upon activation, it should detach from adhesions, either by heat or reaction, and the extraction tool is advanced further until resistance is met. Thus, slowly the sheath is advanced over the lead until it reaches the end where the tip of lead is adhered to the heart wall. At this point the lead is slowly rotated while laser is delivered at an angle to the tip, and the extractor tool is lying outside the heart wall. Thus, without use of much force, the lead is unscrewed and pulled out.

If configuration (ii) is used, then the procedure is vastly similar to one employed above. However the laser delivering optical fibers are mounted on the “assistant tool” and the “extraction tool” is used to deliver force and perform blunt dissection along the plane created by laser activation of the coating as depicted in FIGS. 10A and 10b. FIG. 10A shows the laser light activating the coating having a photosensitive agent and FIG. 10B depicts the retraction of the lead with the extraction device.

In one example with a device where an inactive form of coating (such as photosensitizer or polymer conjugates of photosensitizers that are inactive at implantation), an enzyme can be delivered from the delivery port in the extraction tool to activate this photosensitizer so that it is photo-activatable. Laser is then delivered and causes detachment of external coated layer from its surrounding and extraction performed in a manner explained above.

It should also be noted that in order to protect the normal myocardial or other vascular or adjacent tissue from the effect of photodynamic therapy, or PPTT, or collateral damage from this treatment, quenching photosensitizer molecules may be used.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

1. An implantable device for implantation into tissues of an animal or human comprising:
a. at least one body having an exterior surface for placement in intimate contact with a non-fluid tissue in which said device is implanted;
b. at least one photoreactive agent selected from the group consisting of a nanoparticle and a photosensitizer, which photoreactive agent is associated with said exterior surface, wherein delivery of light energy to said photoreactive agent promotes release of said exterior surface from said tissues in which it is implanted.

2. the implantable device of claim 1 wherein said photoreactive agent is at least one photosensitive nanoparticle in thermal communication with at least one of the group consisting of said exterior surface and the tissue surrounding said exterior surface.

3. The implantable device of claim 1 wherein said body is selected from the group consisting of pacemakers, defibrillators, nephrotomy tubes, indwelling vascular catheters, indwelling neural brain stimulators, indwelling structures, surgical mesh, cochlear implants, dental implants, bladder stimulators, intracardiac monitoring devices, and indwelling stents.

4. The implantable device of claim 3 wherein said body is a pacemaker and said exterior surface is on a cardiac pacing lead.

5. The implantable device of claim 2 wherein said at least one photosensitive nanoparticle is constructed and arranged in association with said exterior surface to cooperate with a source of photons to receive said photons about the exterior surface in intimate contact with said tissue.

6. The implantable device of claim 1 wherein said exterior surface has a therapeutic agent.

7. The implantable device of claim 6 wherein said therapeutic agent is an anti-infective agent.

8. The implantable device of claim 2 wherein said at least one photosensitive nanoparticle is responsive to a selected wavelength of light.

9. The implantable device of claim 8 wherein said selected wavelength of light is produced by a laser.

10. An extraction device for use with an implantable device having at least one body having an exterior surface for placement in intimate contact with non-fluid tissue, said exterior surface having at least one photoreactive agent associated with the exterior surface, said extraction device comprising:
a. an extraction housing having a first end and a second end, said first end having means for manipulation by an operator and said second end for entering an animal or human subject and being positioned proximal to said exterior surface, said second end having a light emitting means for producing light energy; wherein said light energy is received by said photoreactive agent and promotes release of said exterior surface from the tissues in which it is implanted.

11. The extraction device of claim 10 wherein said photoreactive agent is a photosensitive nanoparticle in thermal communication with at least one of the group consisting of said exterior surface and the tissue surrounding said surface, said photosensitive nanoparticle generates thermal energy thermal energy when receiving light energy.

12. The extraction device of claim 10 wherein said extraction housing is a catheter.

13. The extraction device of claim 10 wherein said catheter has a guide lumen.

14. The extraction device of claim 10 wherein said photon emitting means is an optical fiber.

15. The extraction device of claim 14 wherein said optical fiber is in optical communication with a laser.

16. The extraction device of claim 15 wherein said light emitting means is a plurality of optical fibers arranged in a circle about said second end to deliver light around said exterior surface.

17. The extraction device of claim 12 wherein said second end comprises cutting means.

18. The extraction device of claim 17 wherein said cutting means has a substantially circular cutting edge constructed and arranged larger than said exterior surface to allow said cutting edge to cut around the exterior surface.

19. The extraction device of claim 12 wherein said catheter has an extraction sheath for enveloping the exterior surface.

20. The extraction device of claim 12 further comprising a port in fluid communication with one or more liquids to allow administration of such liquids to the location of the exterior surface.

21. A method of extracting an implanted device which is implanted into tissues of an animal or human, said implanted device having at least one body having an exterior surface placed in intimate contact with a non-fluid tissue; said implanted device further comprising at least one photoreactive agent selected from the group consisting of a photosensitizer and a photosensitive nanoparticle wherein delivery of light energy promotes release of said exterior surface from said tissues in which it is implanted, said method comprising the steps of:
a. identifying said exterior surface of said implanted device; and,
b. applying light energy about at least one of the exterior surface and the non-fluid tissue where said implanted device is located to generate thermal energy promoting the release of said exterior surface from said tissue.

22. The method of claim 21 wherein said photoreactive agent is a photosensitive nanoparticle associated with said exterior surface, said photosensitive nanoparticle in thermal communication with at least one of the group consisting of said exterior surface and the tissue surrounding said exterior surface; said photosensitive nanoparticle generates thermal energy upon receiving light energy.

23. The method of claim 21 wherein said body is selected from the group consisting of pacemakers, defibrillators, nephrotomy tubes, indwelling vascular catheters, indwelling neural brain stimulators, indwelling structures, surgical mesh, cochlear implants, dental implants, bladder stimulators, intracardiac monitoring devices, and indwelling stents.

24. The method of claim 21 wherein said body is a pacemaker and said surface is on a cardiac pacing lead.

25. The method of claim 22 wherein said at least one photosensitive nanoparticle is constructed and arranged in association with said exterior surface to cooperate with a source of photons to receive said photons about the exterior surface in intimate contact with said tissue.

26. The method of claim 21 wherein said exterior surface has a therapeutic agent.

27. The method of claim 26 wherein said therapeutic agent is an anti-infective agent.

28. The method of claim 22 wherein said at least one photosensitive nanoparticle is responsive to a selected wavelength of light.

29. The method of claim 28 wherein said selected wavelength of light is produced by a laser.

30. The method of claim 21 wherein an extraction device is used to remove said implanted device, said extraction device having an extraction housing having a first end and a second end, said first end having means for manipulation by an operator and said second end for entering an animal or human subject and being positioned proximal to said exterior surface, said second end having a light emitting means for producing light energy; wherein said light energy is received by said photoreactive agent and promotes release of said exterior surface from the tissues in which it is implanted, said method further comprising the steps of positioning said second end proximal to said exterior surface and using said light emitting means to promote the release of said exterior surface.

31. The method of claim 30 wherein said extraction housing is a catheter.

32. The method of claim 31 wherein said catheter has a guide lumen.

33. The method of claim 30 wherein said light emitting means is an optical fiber.

34. The method of claim 33 wherein said optical fiber is in optical communication with a laser.

35. The method of claim 30 wherein said light emitting means is a plurality of optical fibers arranged in a circle about said second end to deliver light around said exterior surface.

36. The method of claim 30 wherein said second end comprises cutting means and said cutting means is used to cut the exterior surface free of said tissue.

37. The method of claim 36 wherein said cutting means has a substantially circular cutting edge constructed and arranged larger than said exterior surface to allow said cutting edge to cut around the exterior surface.

38. The method of claim 36 wherein said catheter has an extraction sheath for enveloping the exterior surface and said operator cuts around the exterior surface and surrounds the exterior surface with said sheath to grip the exterior surface and withdraw the exterior surface from surrounding tissue.

39. The method of claim 31 wherein said catheter has a port in fluid communication with one or more liquids to allow administration of such liquids to the location of the exterior surface and said method comprises the step of irrigating said exterior surface with one or more liquids from said port.
